(19) United States
(12) Patent Application Publication
(10) Pub. No.: US 2024/0377280 A1
(43) Pub. Date: Nov. 14, 2024

(54) UNIT FOR DETECTING PARAMETERS OF A COMPONENT OF A STRUCTURE AND NETWORK FOR MONITORING A STRUCTURE COMPRISING SUCH UNITS

(71) Applicant: ASDEA SOFTWARE S.R.L., PESCARA (IT)

(72) Inventors: Massimo PETRACCA, PESCARA (IT); Guido CAMATA, PESCARA (IT)

(73) Assignee: ASDEA SOFTWARE S.R.L., PESCARA (IT)

(21) Appl. No.: 18/293,131

(22) PCT Filed: Jul. 21, 2022
(86) PCT No.: PCT/EP2022/070500

§ 371 Date: Jan. 29, 2024

(30) Foreign Application Priority Data
Jul. 29, 2021 (IT) 102021000020231

(51) Int. Cl. G01M 5/00

(52) U.S. Cl. CPC G01M 5/0075; G01M 5/0033

(57) ABSTRACT

A unit for detecting parameters of a component of a structure such as a construction, a building and the like, which comprises at least one processor provided with a signal transceiver, a memory, and connected to a respective electric power supply assembly; at least one sensor, controlled by and connected to the at least one processor. The at least one processor is provided with an adapter module to convert data provided by each sensor into a format that is compatible with the processor.

The present invention relates to a unit for detecting parameters of a component of a structure and to a corresponding network for monitoring a structure comprising such units.

The term structure in the present description is understood to mean any civil or industrial building or architectural and/or engineering element.

Monitoring static parameters, as well as other numerous characteristics of the individual components of any structure, can allow an estimate of the “state of health” thereof, which is understood to be the ability to perform its tasks completely and correctly, as well as to tolerate any extraordinary stresses (such as for example a seismic shock, a large temperature variation, the thrust exerted by a whirlwind, and the like).

The monitoring of the state of health of structures is a particular technique that makes it possible to monitor the state of a construction by using a certain number and type of sensors, placed at key points of the structure.

Knowing the state of health of a structure makes it possible to detect structural damage or fragilities in the early stages, so that it is possible to fix them before they become a serious problem.

All the technical solutions provided in the background art have numerous problems.

Often, they are generally designed only for a specific application and thus, being specifically dedicated to it, cannot be transferred to different structures.

In any case, the background art entails components that are difficult to configure and expensive.

These costs (the complexity of installation and configuration also results in an increase of total costs) limit the adoption of solutions for monitoring the “health” of a construction exclusively to specific and delimited applications.

The aim of the present invention is to solve the problems described above by proposing a unit for detecting parameters of a component of a structure of a versatile type.

Within this aim, an object of the invention is to propose a unit for detecting parameters of a component of a structure of a type that can be modified and configured easily.

Another object of the invention is to propose a unit for detecting parameters of a component of a structure that is easy to obtain and manufacture.

Another object of the invention is to propose a network for monitoring a structure comprising said units which is suitable for any type of construction and/or building.

Another object of the invention is to propose a network for monitoring a structure comprising said units that can be easily updated and re-configured even some time after first installation.

A further object of the present invention is to provide a unit for detecting parameters of a component of a structure and a corresponding network for monitoring a structure comprising said units that has low costs, is relatively easy to provide in practice, and of assured application.

This aim and these and other objects that will become more apparent hereinafter are achieved by a unit for detecting parameters of a component of a structure such as a construction, a building and the like, characterized in that it comprises
at least one processor provided with signal transceiving means, memory means, and connected to a respective electric power supply assembly;
at least one sensor, controlled by and connected to said at least one processor, of the type chosen preferably from an accelerometer, an inclinometer, a gyroscope, a magnetometer, a temperature sensor, a humidity sensor, a strain gauge, a position sensor, a detector of the concentration of specific substances, a detector of electrical fields, a detector of magnetic fields, a vibration detector, a radio wave detector, an optical reader, a GPS locator, and the like;
said at least one processor being provided with an adapter module to convert the data provided by each said sensor into a format that is compatible with said processor.

This aim and these and other objects are also achieved by means of a network for monitoring a structure such as a construction, a building and the like, characterized in that it comprises
at least one unit of the type described in the previous paragraph;
at least one electrical collector provided with a number of connectors suitable for coupling to said units which is not smaller than the number of units that are present in the network and with at least one connector adapted for coupling to a main processing unit designed to process the data that originate from said units;
at least one apparatus for transceiving signals to a component chosen from optionally local display means, a remote server (9), and the like.

Further characteristics and advantages of the invention will become more apparent from the description of a preferred but not exclusive embodiment of the unit for detecting parameters of a component of a structure and of the corresponding network for monitoring a structure comprising said units, illustrated by way of non-limiting example in the accompanying drawings, wherein:

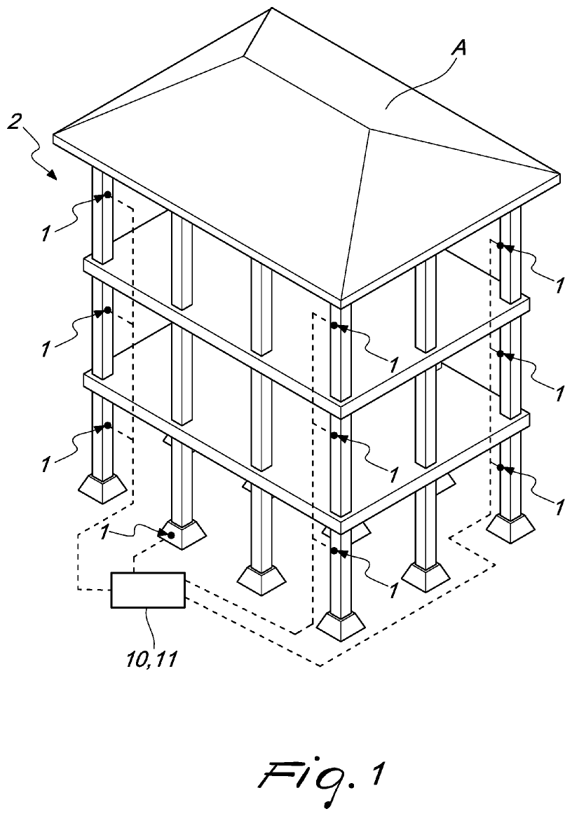

FIG. 1 is a schematic perspective view of a possible embodiment of a network for monitoring a structure such as a construction, a building and the like according to the invention;

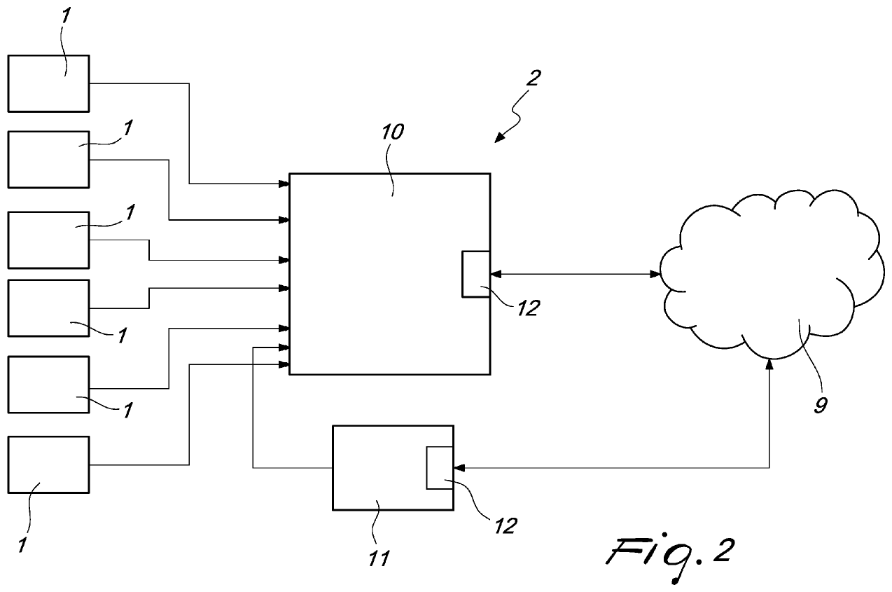

FIG. 2 is a schematic view of a possible embodiment of a network for monitoring a structure such as a construction, a building and the like according to the invention;

FIG. 3 is a schematic view of a unit for detecting parameters of a component of a structure according to the invention;

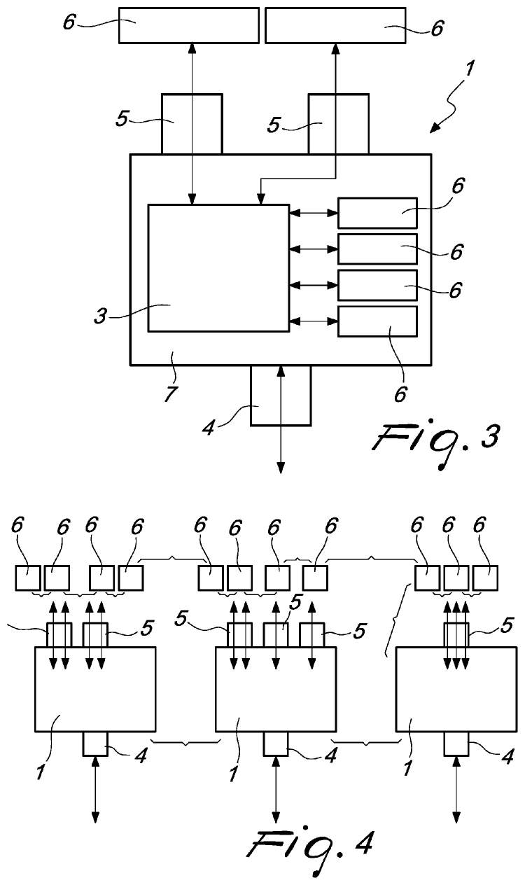

FIG. 4 is a schematic view of some possible interface components that can be associated with a monitoring unit according to the invention.

With particular reference to the figures, the reference numeral 1 generally designates a unit for detecting parameters of a component of a structure and a corresponding network 2 for monitoring a structure A comprising said units 1.

The unit 1 according to the invention comprises at least one processor 3 provided with signal transceiving means 4, memory means, and connected to a respective electric power supply assembly. With particular reference to the diagram shown in FIG. 3, the signal transceiving means 4 can be incorporated in a single component dedicated to connection to the outside: in practice, said component allows connection to an interconnected circuit for communication and data transmission (for example, an Ethernet network) and also to an electric power supply source (which for example might be the electrical network that exists in the structure A in which the unit 1 will be installed, optionally with the interposition of devices designed to adapt the electric power supply parameters).

The unit 1 further comprises at least one sensor 6, controlled by and connected to the at least one processor 3. The sensor 6 is of the type chosen preferably from an accelerometer, an inclinometer, a gyroscope, a magnetometer, a temperature sensor, a humidity sensor, a strain gauge, a position sensor, a detector of the concentration of specific substances, a detector of electrical fields, a detector of magnetic fields, a vibration detector, a radio wave detector, an optical reader, a GPS locator, and the like.

Many of the detectors cited as possible components of the unit 1 (which may constitute one of the sensors 6) may be of a different type, depending on the technology used for the detection of the parameter of interest and also according to the research methodology.

For this reason, it is specified that the accelerometers, inclinometers, gyroscopes, magnetometers, position sensors, detectors of electrical fields, detectors of magnetic fields, vibration detectors, radio wave detectors may be uniaxial or multiaxial (in particular triaxial for the applications of interest).

It is specified that according to the invention the at least one processor 3 may be advantageously provided with an adapter module to convert the data provided by each sensor 6 into a format which is compatible with such processor 3.

One possible variation of the unit 1 according to the invention may comprise at least one module 5 for connection to external components.

With particular reference to an embodiment of unquestionable interest in practice and in application, each unit may conveniently comprise an accommodation support 7 for the at least one processor 3, the signal transceiving means 4, the memory means, the at least one sensor 6 (which is of the type preferably chosen from an accelerometer, an inclinometer, a gyroscope, a magnetometer), the at least one module 5 for connection to 20 external components (which are preferably chosen from a temperature sensor, a humidity sensor, a strain gauge, a position sensor, a detector of the concentration of specific substances, a detector of electrical fields, a detector of magnetic fields, a vibration detector, a radio wave detector, an optical reader, a GPS locator, and the like).

The support 7, in most of the practical applications of applicative interest, accommodates at least two sensors 6 chosen from an accelerometer, an inclinometer, a gyroscope, and a magnetometer. In particular, FIG. 6 shows a unit 1 in which the associated support 7 accommodates an accelerometer of the triaxial type, an inclinometer of the triaxial type, a gyroscope of the triaxial type, and a magnetometer of the triaxial type. This constructive configuration is the one that allows the best monitoring (in terms of assessment of static performance and structural rigidity and strength) of the structure A with which the unit 1 is associated.

Moreover, it is noted that the at least one connection module 5 can favorably comprise at least one respective connector for the wiring of at least one external component.

Depending on the specific type of installation provided and the respective requirements for monitoring specific parameters, the number of connection modules 5 of each unit 1 may be varied, adapting it to the number of sensors 6 that one intends to connect to said unit 1. Each module 5 is configured to adapt to the communication protocol of the specific sensor 6 with which it is associated (for example a parallel transmission instead of a serial one, or an analog transmission instead of a digital one, combinations of said cases, and the like).

Furthermore, it is noted that the signal transceiving means 4 may be advantageously connected to a network of a type chosen from a wired network of the LAN type, a wireless network of the WLAN type, a combination thereof, and the like.

In this manner, the data processed and collected by the unit 1 may be shared with users who are anywhere, or may be uploaded to a remote server (for example a cloud 9) or examined in situ by virtue of specific display means.

The users may view the data collected by each unit 1 directly on their personal computer or on their smartphone or tablet or generic connected device.

Furthermore, it is appropriate to note that the memory means associated with the processor 3 are adapted to record and collect software of the type chosen from drivers of sensors 6, applications for the control and management of the sensors 6, protocols for the conversion of the data strings that originate from the sensors 6 into equivalent strings compatible with the operating language of the processor 3, applications for the transceiving of signals externally on the part of the processor 3, and the like. In practice, the memory means may store all the firmware that allows the correct operation of each sensor 6 and its correct control by the processor 3.

Moreover, the present invention extends its protection also to a monitoring network 2 of a structure A such as a construction, a building, and the like, which comprises at least one unit 1 of the type described so far.

Such network 2, according to the invention, comprises conveniently at least one electrical collector 10 provided with a number of connectors adapted for coupling to the units 1 that is not smaller than the number of units 1 present in the network 2. Furthermore, there is also an additional connector adapted for coupling to a main processing unit 11 designed to process the data that originate from all the units 1.

At least one signal transceiving apparatus 12 is also associated with the collector 10 and may interface with local means for display (data inspection and monitoring) or with a remote server 9.

The alternative possibility is provided to associate the transceiving apparatus 12 with the main processing unit 11, so as to allow it to interface with the remote server or with any other enabled device that can be used by a specific user of the network 2.

The adoption of two transceiving apparatuses 12, one associated with the collector 10 and one associated with the main processing unit 11, in order to increase the convenience of use of the network 2, is also not excluded.

It is specified that the connectors of the electrical collector 10 are of a type chosen preferably from mechanical, for the connection of respective cables that originate from a component chosen from a unit 6 and the main processing unit 11, and of the wireless type, for connection to a component chosen between a unit 6 and the main processing unit 11 by means of a respective transceiving protocol.

The main processing unit 11 is advantageously preset to process the data that originate from the units 1 and comprises advantageously a memory element containing a model of TCP/IP protocols for communication with the server 9 and with any additional remote devices of a type chosen from smartphones, tablets, computers, and the like.

Furthermore, it is specified that the network 2 may positively have an information technology architecture according to which the main processing unit 11 is configured as master and all the units 1 are configured as slaves.

In this manner, the clock pulses of all the units 1 are synchronized with the clock pulses of the main processing unit 11.

An embodiment in which a specific unit 1 is configured as master and in which all the other units 1 of the network 2 are configured as slaves (and therefore synchronized therewith) is not excluded.

Knowing the state of health of a structure A allows the detection of structural damage or fragilities in the early stages of their development, so that it is possible to intervene for a respective restoration, before they can become a serious problem.

The adoption of the network 2 according to the invention produces three positive results:
1. it allows saving time, since repairing damage in its initial phase usually requires a smaller amount of labor and time and generally can be performed without interrupting the operation of the structure A;
2. it allows saving money, because repairing damage as soon as it occurs (and therefore when it is still minimal) is generally much cheaper than repairing it when it has spread and involves a considerable portion of said structure A;
3. it allows safeguarding the safety of the people who use the structure A, since knowing its state of health is essential to ensure safety; if a structure A is in a good state of health, the probabilities that it withstands external stresses, even extraordinary one such as earthquakes, are much higher.

A structural monitoring network 2 is substantially composed of a predefined number of units 1 spread throughout the structure A (as shown schematically in FIG. 1). All the data originating from the units 1 can be collected by the main processing unit 11, analyzed and conveniently coordinated in order to estimate the state of health of the structure A.

The unit 1 collects the raw data.

The quality of the final result of the estimate of the structural health of the structure A depends greatly on two factors: the quality of the processing algorithm and the quality of the assembly constituted by the individual units 1 and their mutual interconnections.

The quality of the processing algorithm depends mainly on the placement of the units 1 in the structure A and on the algorithm that processes the raw data. It may also depend on the number of units 1, since having a large number of units 1 can be a key factor.

The quality of the network 2 depends on:
the characteristics of each unit 1 and in particular the number and type of sensors 6 comprised in each one of them;
the number of units 1 installed in relation to the maximum number of units that can be managed by the network 2;
the accessibility of the data, understood to be the ease of access to the data for the end user;
the price.

The structures A can be composed of various materials, with different techniques, and it is not uncommon to find different materials and techniques on the same structure A.

Different types of structure A require different types of sensors 6.

This is a problem for units of the standard type, since each one would have to include the right sensor for each situation.

For example, a structure made of concrete may require just an accelerometer, a structure made of wood may also require a hygrometer in order to measure the humidity of the wood, et cetera.

The maximum number of units 1 supported by the network 2 is often a problem for currently commercially available solutions. One particular type of structure A, particularly large structures A (a large building), requires a large number of sensors 6, even on the order of hundreds, and in most cases all the sensors 6 have to share an absolute time reference in order to maintain consistency among the samples collected by the respective units 1. This is one of the most difficult problems to overcome in a structural monitoring network 2.

The problem related to the accessibility of the data is particularly important, especially in the current historical period, where everything is moving toward IoT and Industry 4.0.

The connectivity between the monitoring network 2 and the outside world is fundamental for the ease of remote access to the data both by the processing algorithm and by the end user.

The use of technologies of the standard type for the provision of the network 2 and of the units 1, especially with regard to connectivity, makes it possible to reduce its costs significantly.

Currently commercially available solutions are not capable of achieving satisfactory results.

The unit 1 according to the invention instead meets all the requirements described above, particularly by virtue of the fact that each unit 1 behaves as an intelligent node of the network 2, being capable of performing edge computing on the collected data, being able to detect events, being able to change its own characteristics (such as for example, the sampling frequency, the scale of the sensors 6, et cetera), and being able to automatically recognize new sensors 6 connected thereto.

Moreover, the unit 1 according to the invention has high adaptability (versatility): in fact, it comprises a certain number of sensors 6 integrated on the support 7, but has modules 5 (the number of which may optionally be modified as a function of the specific requirements) in order to connect additional sensors 6 preferably in a manner of the plug-and-play type.

Each unit 1 is a device that falls within the technology termed IoT (Internet of Things); in fact, it can connect to the Internet, being therefore remotely accessible by the user.

Therefore, it is possible to deem each individual unit 1 to be a complete electronic system.

The main element of the unit 1 is the processor 3 (controller), which is capable of:
interfacing with internal devices by means of a common inter-IC digital bus, such as I2C or SPI;
interfacing with external devices and/or with external devices and sensors 6 with a common digital bus among devices (for example RS-232/RS-422) and supplying power to them;
interfacing with external devices and/or external devices/sensors 6 that require an analog interface (for example, a humidity sensor in which the value is measured as a variation in the resistance of a resistor and this requires an analog port to measure the resistance) and supplying power to them;
connecting over a PoE Ethernet line at at least 10 Mbps (optimum results are expected by adopting a line at 100 Mbps or higher, although it is not excluded, for specific applications, to use lines with even much lower performance);
having sufficient computing power to manage the other connected devices (for example, the sensors 6), both integrated on the support 7 and external, performing edge computing on the collected data before sending it over Ethernet (for example, event detection), managing Ethernet and remote connection, managing PTP, the Precision Time Protocol, as a slave or master, and synchronizing the clock with a master, so that each node shares the same absolute time.

Almost all structural health monitoring applications require a basic set of sensors: an accelerometer (operating on 1 or more axes), an inclinometer (operating on 1 or more axes), and a gyroscope (operating on 1 or more axes).

Since this set is so common in the world of structural monitoring, each unit 1 by default conveniently contains any combination of these sensors, meaning that each unit 1 can contain just one or all three. Furthermore, each node may optionally comprise a magnetometer (also operating on 1 or more axes).

The magnetometer may be used together with the accelerometer in order to detect the spatial orientation of the unit 1.

If the particular monitoring application requires multiple sensors 6, it is possible to connect them by means of the modules 5, which can adapt to the characteristics of the sensor 6 and are sufficient in number to ensure the connection of all the sensors 6 needed.

Therefore, an unspecified number of modules 5 may be present on each unit 1. The modules 5 may be of the digital type, in order to communicate with sensors that communicate with a particular digital bus (for example, RS232, RS422, I2C, SPI, etcetera), or of the analog type, in order to acquire data from analog sensors (for example, measure a resistance directly).

The module 5 for connection to external components comprises connectors that can provide any type of mechanical interconnection (for example, M12, M16, cable gland, et cetera).

The modules 5 (and the possibility to configure the unit 1 with an indefinite number of them) can allow the unit 1 to best adapt to the most disparate installations: for example, measuring wood humidity, monitoring the position of particular points by means of GPS, monitoring the deformation of critical parts in the structure or the distance between piers or the fractures by using strain gauges, et cetera.

Each module 5 may also be simultaneously digital and analog and support multiple communication protocols at the same time, since electronically it is possible to automatically determine the type of sensor 6 connected and reconfigure the module 5 using the internal electronics of the unit 1.

The onboard intelligence (the processor 3 and its memory means) contains all the drivers and applications for all possible connectable sensors 6, so that the installation technician only has to connect the new sensor 6, which will start operating automatically.

If operation according to plug-and-play logic is not possible for a specific sensor 6 (the unit 1 does not recognize the sensor 6), the installation technician may communicate directly to the unit the type of sensor 6 connected and the unit 1 will automatically start acquiring the corresponding data.

The requirements that external sensors 6 must have in order to be validly associated with the unit 1 are as follows: compatibility with the mechanical shape of one of the connectors of the module 5 with which they are to be associated; requiring a power supply lower than or equal to the maximum power supply that can be delivered by the unit 1, or being suitable for connection to a respective external power supply.

In this manner, the cost of the unit 1 is kept low and additional sensors 6 can be installed only for those applications in which they are needed, making the units 1 and the network 2 that comprises them an extremely economical and easily installable solution.

The communication interface of the unit 1 with the outside world can advantageously be an at least 10-Mbps Ethernet (a high-performance line allows each unit 1 and the network 2 to operate more efficiently, although Ethernet lines with lower performance are not excluded in the case of specific applications) with a full IP stack. This makes it possible to connect the unit 1 on a private LAN or to the Internet in order to then access it remotely. The advantages that reside in this particular embodiment are: a reduction in costs, since Ethernet is a widely used communication protocol and is extremely cost-effective; scalability, since the units 1 can be connected to or disconnected from the network 2 at any time since everything is plug and play; data accessibility, since Ethernet easily allows remote connection to each individual unit 1 substantially at any time; the use of the Power over Ethernet (POE) technology, so that with a single cable it is possible to both communicate with and supply power to the node (by means of the module 4).

The only drawback of Ethernet with an IP stack is the fact that it is not deterministic and therefore might lead to a loss in time consistency among units.

In order to obviate this problem, all the units 1 run the PTP protocol (IEEE 1588) with a slave configuration.

In this manner, all the units 1 synchronize their clocks with the same master (which might be the one of the processor 11 (ββ) or of a specific conveniently configured unit 1 or of an additional device not shown in the present description), so that all the units 1 share the same absolute time with sub-millisecond precision.

In this manner, temporal consistency among the data collected by the units 1 is ensured.

Advantageously, the present invention solves the problems described above, proposing a unit 1 for detecting parameters of a component of a structure A of a particularly versatile type.

Conveniently, the unit 1 according to the invention is of an easily modifiable and configurable type.

Positively, the unit 1 according to the invention is constituted by components that are easy to find and build.

Usefully, the monitoring network 2 of a structure A comprising such units 1 is suitable for any type of construction and/or building.

Advantageously, the network 2 according to the invention can be updated and re-configured easily even some time after first installation.

Validly, the unit 1 and the corresponding monitoring network 2 according to the invention are relatively simple to provide in practice and have low costs: these characteristics make the unit 1 and the network 2 according to the invention innovations of assured application.

The invention thus conceived is susceptible of numerous modifications and variations, all of which are within the scope of the appended claims; all the details may furthermore be replaced with other technically equivalent elements.

In the examples of embodiment shown, individual characteristics, given in relation to specific examples, may actually be interchanged with different characteristics that exist in other examples of embodiment.

In practice, the materials used, as well as the dimensions, may be any according to the requirements and the state of the art.

The disclosures in Italian Patent Application No. 102021000020231 from which this application claims priority are incorporated herein by reference.

1.-11. (canceled)

12. A unit for detecting parameters of a component of a structure such as a construction, a building and the like, comprising
at least one processor provided with signal transceiving means, memory means, and connected to a respective electric power supply assembly;
at least one sensor, controlled by and connected to said at least one processor, of a type chosen preferably from an accelerometer, an inclinometer, a gyroscope, a magnetometer, a temperature sensor, a humidity sensor, a strain gauge, a position sensor, a detector of a concentration of specific substances, a detector of electrical fields, a detector of magnetic fields, a vibration detector, a radio wave detector, an optical reader, a GPS locator, and the like;
said at least one processor being provided with an adapter module to convert data provided by each said sensor into a format that is compatible with said at least one processor.

13. The unit according to claim 12, wherein said at least one processor comprises at least one connection module for connection to external components.

14. The unit according to claim 13, further comprising an accommodation support for
said at least one processor,
said signal transceiving means,
said memory means,
said at least one sensor of a type chosen preferably from an accelerometer, an inclinometer, a gyroscope, a magnetometer,
said at least one connection module for connection to external components chosen preferably from a temperature sensor, a humidity sensor, a strain gauge, a position sensor, a detector of a concentration of specific substances, a detector of electrical fields, a detector of magnetic fields, a vibration detector, a radio wave detector, an optical reader, a GPS locator, and the like.

15. The unit according to claim 14, wherein said support accommodates at least two sensors chosen from an accelerometer, an inclinometer, a gyroscope, and a magnetometer.

16. The unit according to claim 13, wherein said at least one connection module comprises at least one respective connector for a wiring of at least one external component.

17. The unit according to claim 12, wherein said signal transceiving means are connected to a network of a type chosen from a wired network of the LAN type, a wireless network of the WLAN type, a combination thereof, and the like.

18. The unit according to claim 12, wherein said memory means are configured to record and collect software of a type chosen from sensor drivers, applications for control and management of the sensors, protocols for a conversion of data strings that originate from the sensors into equivalent strings compatible with an operating language of said processor, applications for transceiving of signals externally on the part of said processor, and the like.

19. A network for monitoring a structure of a type of a construction, a building, and the like, comprising
at least one unit according to claim 12;
at least one electrical collector provided with a number of connectors configured for coupling to said at least one unit which is not smaller than a number of units that are present in the network and with at least one connector configured for coupling to a main processing unit designed to process data that originate from said units;
at least one apparatus for transceiving signals to a component chosen from optionally local display means, a remote server, and the like.

20. The monitoring network according to claim 19, wherein said connectors of said electrical collector are of a type chosen preferably from mechanical, for the connection of respective cables that originate from a component chosen from one of said units and said main processing unit, of a wireless type, for connection to a component chosen between one of said units and said main processing unit by means of a respective protocol.

21. The monitoring network according to claim 19, wherein said main processing unit designed to process the data that originate from said units comprises a memory element which contains a model of TCP/IP protocols for communication with said server and with any additional remote devices of a type chosen from smartphones, tablets, computers, and the like.

22. The monitoring network according to claim 19, having an information technology architecture according to which one component chosen from said main processing unit and one unit is configured as master and all the other units are configured as slaves, clock pulses of all of said units being synchronized with clock pulses of said main processing unit.
